SEQUENCE LISTING (2 sequences)

SEQ ID NO: 1 - DNA, 24 bp, Artificial Sequence
  Description of Artificial Sequence oligonucleotide
tccagaatctgttccagagcgtgc

SEQ ID NO: 2 - DNA, 24 bp, Artificial Sequence
  Description of Artificial Sequence oligonucleotide
gctgtgaaggttgctgttcctcat

(19) United States
(12) Patent Application Publication
(10) Pub. No.: US 2006/0040263 A1
(43) Pub. Date: Feb. 23, 2006

(54) Marker at the androgen receptor gene for determining breast cancer susceptibility

(75) Inventor: Francois Rousseau, Sainte-Foy (CA)

Correspondence Address: FOLEY & LARDNER LLP, P.O. BOX 80278, SAN DIEGO, CA, 92138-0278, US

(73) Assignee: SignalGene, Inc.

(21) Appl. No.: 10/445,214

(22) Filed: May 23, 2003

Related U.S. Application Data
(63) Continuation of application No. 09/787,493, filed on Jul. 12, 2001, now abandoned.
(60) Provisional application No. 60/100,311, filed on Sep. 15, 1998.

(51) Int. Cl. C12Q 1/68; C12P 19/34

(52) U.S. Cl. 435/6; 435/91.2

(57) ABSTRACT

The present invention relates to a method of determining an individual's predisposition to breast cancer, development of breast cancer, protection against breast cancer and/or responsiveness to therapy for breast cancer. The method comprises the step of determining the androgen receptor genotype at the CAG repeat locus of an individual, or at a locus in linkage disequilibrium with the CAG repeat locus, thereby determining an individual's predisposition to breast cancer, development of breast cancer, protection against breast cancer and/or responsiveness to therapy for breast cancer.

Breast cancers have the same clinical characteristics In older as in younger women. Cancer is usually suspected when changes are noted on mammography or when a breast lesion is seen or felt. Lesions usually can be felt as firm nodules within the breast. Ulcerations may occur, and lesions within or near the nipple may produce discharge. Sometimes breast cancer is discovered only after metastatic lesions cause bone fractures, neurologic changes, hypercalcemia, liver failure, or ascites.

When a tumor is detected by physical examination, bilateral mammograms are normally obtained to rule out occult lesions. Certain radiographic images, such as speckled calcifications or tissue infiltration, suggest cancer, while a cystic appearance suggests a benign process. Even an apparently benign finding on mammogram requires further evaluation. Generally the diagnosis is established by fine needle aspiration. Fine needle aspiration allows collection and cytological examination of cystic fluid and is helpful In planning definitive treatment of breast cancer. Although a positive result on fine needle aspiration is diagnostic, a negative result is usually followed by an open biopsy. Now a day, there is still no specific test for assaying predisposition or resistance to breast cancer.

Since the discovery of the human androgen receptor (AR) gene, mutations in this gene have been associated with Kennedy's disease (spinobulbarmuscolar atrophy), with various degrees of androgen insensitivity and with prostate cancer.

Elhaji et al. (American Journal of Human Genetics, vol. 61, no. 4 suppl, 28.10.1997-1.11.1997, page A64) assesses the distribution of CAG-repeat length of the AR in breast cancer tissue to evaluate the possible correlation between the repeat length and the risk of breast cancer. However, Elhaji et al. is concerned with breast cancer tissue per se and not with the potential of using AR as a marker for determining the predisposition and prognosis of breast cancer by, for example, screening patients prior to the development of breast cancer. Indeed, Elhaji et at. suggests that somatic mutations are involved In shifting the distribution of the CAG repeat of the AR gene and thus, that a predisposition and prognosis test could not be carried-out.

Thus, an association between a gemline mutation in AR gene and predisposition to breast cancer has yet to be reported.

There thus remains a need to provide a genetic assay for determining the predisposition and/or resistance to breast cancer development of breast cancer and responsiveness to therapeutic modalities.

While some markers have been identified as genetic determinants for breast cancer and/or as risk factors to develop same (i.e. BRCA1 and BRCA2), there remains a need to identify new markers therefor. More specifically, there remains a need to provide means to determine a predisposition to breast cancer and/or responsiveness to therapy to breast cancer, by analyzing allelic variations in genes associated with breast cancer. In addition there remains a need to identify patients who are likely to benefit from a particular prevention or therapeutic treatment program. Further, there remains a need to provide assays to screen for compounds (i.e. hormones, molecules acting on hormone receptors or other agents) that could be beneficial to patients.

The present invention seeks to meet these and other needs.

The present description refers to a number of documents, the content of which is herein incorporated by reference, in their entirety.

### SUMMARY OF THE INVENTION

One aim of the present invention is to provide a genetic assay for determining the predisposition to breast cancer and/or response to breast cancer treatment.

Another aim of the present invention is to use a polymorphism of the androgen receptor (AR) gene or an equivalent thereof as a marker for breast cancer susceptibility and/or response to breast cancer preventive or curative therapy. A polymorphism of the androgen receptor (AR) gene, or any polymorphism in linkage disequilibrium therewith, can be used as a test for breast cancer susceptibility, for responsiveness to treatment of breast cancer, for breast cancer prognosis or severity, or as a means to classify patients in clinical trial for breast cancer (screening, diagnosis, prognosis or treatment).

One of a polymorphism of the AR gene, or any polymorphism in linkage disequilibrium therewith, can further be used as a test for screening drugs for breast cancer or for determining the best treatment therefor.

Broadly, the present invention aims at providing a method of determining the length of a CAG repeat polymorphism in the androgen receptor gene, wherein this determination can be correlated with a predisposition or a protection to breast cancer. This determination can be based on a variety of genotyping methods at the DNA, RNA or protein level.

Another aim of the present invention is to provide a method of prognosing and/or forecasting the development of breast cancer in a patient, which comprises determining a CAG-repeat polymorphism of the AR gene, or any polymorphism in linkage disequilibrium therewith, in a biological sample of the patient, wherein a determination of the length of the CAG repeat shows a significant association with breast cancer.

In a particular embodiment, the determination of the polymorphism at the CAG repeat of the AR gene enables to show that the shortest alleles or a combination of the shortest alleles are associated with the smallest breast cancer risk and the mid to long alleles or a combination of the intermediate and longest alleles are associated with the highest breast cancer risk (a combination of the longest alleles is associated with the highest risks of breast cancer). Of importance, the variations of polymorphisms at the CAG repeat locus of AR (or of an equivalent or marker in linkage disequilibrium therewith) can account for a significant proportion of all cases of breast cancer. Indeed, the number of breast cancer cases attributable to a variation at this AR locus is at least three times greater than that attributable to the BRCA1 and BRCA2 genes.

The present invention also relates to vectors, including expression vectors harboring an AR gene (or fragment or fusion thereof) having a genotype in accordance with the present invention (i.e. a predisposing genotype, long CAG repeats, or alternatively, a protecting genotype, short CAG repeats; or other genotypes isolated from patients or genetically engineered), cells harboring such vectors, and non-human animals harboring such vectors or cells.

Another aim of the present invention is to provide means of identifying young women that will be at risk of developing breast cancer and to categorize those that are likely to respond significantly to preventive therapy. An aim of the present invention is thus to provide means of identification of target sub-groups of women for breast cancer prevention measures/programs.

Another aim of the present invention is to provide means to determine which sub-group of women will most benefit from breast cancer treatment(s) and eventually predict their response to therapy or choose the optimal preventive pharmacotherapy.

Another aim of the present invention is to identify means of predicting and managing interventions for breast cancer as well as identifying and/or characterizing biological parameters which could enable the establishment of population-based breast cancer prevention and intervention programs.

In addition, it is an aim of the present invention to provide a method of selecting alleles of the AR gene or in linkage disequilibrium therewith, which is suitable for designing an assay to screen compounds which can modulate the activity of an androgen receptor.

Another aim of the present invention is to provide an assay to screen for drugs for the treatment and/or prevention of breast cancer. Having identified alleles which predispose to breast cancer (and those which predispose to a “resistance” to breast cancer), assays can be set-up to screen agents and select drugs which could be used in the treatment or prevention of breast cancer. Since some alleles of the AR have been shown to affect the functionality of the androgen receptor (Tut et al. 1997, J. Clin. Endocrinol. 89(11):3777-3782), assays could be designed based on chosen genotypes of the AR gene. A non-limiting example of a type of assay which could be designed includes, cis-trans assays similar to those described in U.S. Pat. No. 4,981,784. For example, a cis-trans assay could be set-up, based on the use of a genotype of AR, shown here to predispose to breast cancer (i.e. the long CAG alleles in the AR gene) as compared to a genotype of AR, shown here to be associated with lower risk of breast cancer, and used to screen compounds. A non-limiting example of such an assay could be based on 2 cell lines (one expressing a predisposing genotype of AR and one expressing a non-predisposing genotype of AR) which could be used in parallel to screen for AR-function modulating compounds. Of course, it will be understood that the cell line expressing the non-predisposing genotype of AR (the shorter alleles) can be used as a positive control for the functionality of the androgen receptor.

It is thus an aim of the present invention to provide the means to identify compounds which could positively modulate the function of AR having a breast cancer predisposing genotype (such as the long CAG alleles), to the level of the protecting genotype thereof (such as the short CAG alleles).

In a particular embodiment, such assays can be designed using cells from patients having a known genotype at the loci of the present invention, these cells harboring recombinant vectors could enable an assessment of the functionality of the AR and dissect the structure-function relationship of the androgen receptor and its role in breast cancer.

It shall be understood that the polymorphism of the AR and/or the determination of allelic variations in the AR gene can be combined to the determination of allelic variations in other genes/markers linked to the predisposition to breast cancer and/or responsiveness to therapy therefor. This combination of genotype analyses could lead to better diagnoses programs and/or treatment of breast cancer. Non-limiting examples of such markers include BRCA1 and BRCA2.

It shall also be understood that although breast cancer is significantly more preponderant in women, it can also be a deadly disease in men. Thus, the present invention is meant to also cover men.

In accordance with the present invention, there is therefore provided a method of determining an individual's predisposition to breast cancer, development of breast cancer and/or responsiveness to therapy for breast cancer, which comprises determining a genotype at the CAG-repeat locus of the androgen receptor (directly or indirectly by linkage disequilibrium) in a biological sample of the individual and analyzing allelic variation in the androgen receptor of the individual, thereby determining an individual's predisposition to breast cancer, development of breast cancer and/or responsiveness to therapy therefor.

In accordance with the present invention there is provided a method for determining susceptibility to breast cancer, and/or response to therapy therefor. The method comprises the step of determining the androgen receptor genotype of the individual, thereby determining an individual's susceptibility to breast cancer and/or response to therapy therefor.

Numerous methods for determining a genotype are known and available to the skilled artisan. All these genotype determination methods are within the scope of the present invention. Non-limiting examples of genotype determination include a restriction endonuclease digestion, a hybridization with allele specific oligonucleotides, a sequencing of the polymorphism, and an amplification of a segment of the androgen receptor (i.e. by PCR).

In accordance with the present invention, there is therefore provided a method of determining an individual's predisposition to breast cancer, development of breast cancer and/or responsiveness to therapy therefor, which comprises determining androgen receptor polymorphism (directly or indirectly using a marker in linkage disequilibrium with the CAG repeat polymorphism) in a biological sample of the individual and analyzing allelic variation in the androgen receptor gene of the individual, thereby determining an individual's predisposition to breast cancer, development of breast cancer and/or responsiveness to therapy therefor.

In accordance with one embodiment of the invention, there is provided a specific model for use in prediction of breast cancer susceptibility and prognosis. The model comprises an androgen receptor gene polymorphisms at the CAG repeat locus, that allows to identify a subset of women that are at significantly increased risk of breast cancer as compared to those bearing other variant of this gene.

In accordance with a preferred embodiment of the present invention, a single gene, the androgen receptor gene, has been identified as such a target to assess this predisposition.

In accordance with the present invention, the androgen receptor polymorphism, without limitation, is selected from the CAG repeats located in the first exon of the AR gene, or any DNA variant or mutation which shows some degree of linkage disequelibrium with one of the polymorphisms at the CAG-repeat locus of the AR gene.

In some embodiments, the method of the present invention includes detecting the androgen receptor polymorphism by analyzing the restriction fragment length polymorphisms using an endonuclease digestion. The method can further include a step prior to the androgen receptor gene digestion, wherein at least a fragment of the androgen receptor is amplified, for example, by polymerase chain reaction.

In accordance with a preferred embodiment of the present invention, a pair of primers is designed to specifically amplify a segment of the androgen receptor. In an especially preferred embodiment, the region of the AR gene which is amplified is in exon 1. This pair of primers is preferably derived from a nucleic acid sequence of the androgen receptor gene or flanking portion thereof, to amplify a segment of the androgen receptor gene, as commonly known. Of course, other primer pairs can be designed, based on the known sequence of the AR gene. Method to design primer pairs form known sequences are commonly known in the art.

In accordance with a preferred embodiment of the present invention, primers used for amplifying the segment of the androgen receptor are defined as:

| | |
|---|---|
| 5′-TCCAGAATCTGTTCCAGAGCGTGC-3; | (SEQ ID NO: 1) |
| and | |
| 5′-GCTGTGAAGGTTGCTGTTCCTCAT-3′. | (SEQ ID NO: 2) |

The polymorphism of the androgen receptor gene can be detected using at least one oligonucleotide specific to the normal or variant androgen receptor gene allele.

The present invention also provides a kit for determining predisposition to low, intermediate or high risk of breast cancer of a patient, which includes at least a probe specific for the androgen receptor; a polymorphism selected from the group consisting of a CAG repeat and other polymorphisms in linkage disequilibrium with the CAG repeat polymorphism.

In one embodiment, the present invention provides a specific detection of the CAG repeat polymorphism of the AR gene using a nucleic acid for the specific detection of this AR polymorphism in a sample comprising the above-described CAG-repeat-containing nucleic acid sequence (i.e. DNA, RNA, cDNA) and at least a nucleic acid sequence which binds under stringent conditions to the CAG-repeat-containing nucleic acid sequence.

In one prefered embodiment, the present invention relates to nucleic acid probes which are complementary to a CAG-repeat-containing nucleic acid sequence, consisting of at least 10 consecutive nucleotides (preferably, 15, 20, 25, or 30) and which specifically hybridize to the AR nucleic acid sequence comprising the CAG repeat polymorphism under high stringency condition.

In one embodiment of the above described method, a nucleic acid probe is immobilized on a solid support. Non-limiting examples of solid supports include plastics (i.e. polycarbonate), acrylic resins (i.e. polyacrylamide and latex beads); and carbohydrates (i.e. agarose and sepharose). Techniques: for coupling nucleic acid probes to solid supports are well known in the art.

Similarly to the probes of the present invention, the antibodies of the present invention can be immobilized on a solid support. As known in the art, similar supports as those used for probe immobilization can be used for antibody immobilization on a solid support. Also well known in the art are the techniques for coupling antibodies to such solid supports. The immobilized antibodies of the present invention can be used for in vitro, in vivo, and in situ assays as well as in immunochromatography according to known methods.

Non-limiting examples of test samples suitable for carrying the methods of the present invention include, cells or nucleic acid extracts of cells, or biological fluids. Of course, the type of test sample used can vary according to the assay format, the method of detection, and the particular needs of the clinical practioner which will readily adapt the methods of preparation of the sample and the method of detection so that they are compatible, in accordance with the knowledge in the art.

In accordance with one embodiment of the present invention, the allelic variation in the androgen receptor gene is analyzed indirectly using a nucleic acid variant, or equivalent in linkage disequilibrium with a CAG repeat. The allelic variation in the androgen receptor gene can also be analyzed directly by determining the number of CAG repeat within the androgen receptor gene.

In accordance with the present invention, the polymorphism of the androgen receptor (AR) gene can be used as a marker for breast cancer susceptibility. The polymorphism in linkage disequilibrium with the markers used can also be used as a test for breast cancer susceptibility, or for responsiveness to treatment for breast cancer, for breast cancer prognosis or severity, or as a means to classify patients in clinical trials for breast cancer (screening, diagnosis, prognosis or treatment).

In order to provide a clear and consistent understanding of terms used in the present description, a number of definitions are provided hereinbelow.

As used herein the term “RFLP” refers to restriction fragment length polymorphism.

The terms “polymorphism”, “DNA polymorphism” and the like, refer to any sequence in the human genome which exists in more than one version or variant in the population.

The term “linkage disequilibrium” refers to any degree of non-random genetic association between one or more allele(s) of two different polymorphic DNA sequences, that is due to the physical proximity of the two loci. Linkage disequilibrium is present when two DNA segments that are very close to each other on a given chromosome will tend to remain unseparated for several generations with the consequence that alleles of a DNA polymorphism (or marker) in one segment will show a non-random association with the alleles of a different DNA polymorphism (or marker) located in the other DNA segment nearby. Hence, testing of one of a marker in linkage desiquilibrium with the polymorphisms of the present invention at the AR gene (indirect testing), will give almost the same information as testing for the CAG repeat polymorphism of the AR gene directly. This situation is encountered throughout all the human genome when two DNA polymorphisms that are very close to each other are studied. Such a linkage disequilibrium has been reported with several polymorphisms in several genes (i.e. the vitamin D receptor gene [Morrisson et al., 1994, Nature 367:284-287, and U.S. Pat. No. 5,593,033]). Various degrees of linkage disequilibrium can be encountered between two genetic markers so that some are more closely associated than others.

The terms “androgen receptor polymorphism” or “genetic marker” are intended to include, without limitation, the CAG-repeat polymorphism in exon 1, and any other allelic variant of the androgen receptor gene that show some degree of linkage disequilibrium in any population sub-group, with at least one of the above-mentioned androgen receptor polymorphisms.

The androgen receptor gene polymorphism sites in accordance with the present invention can be located within the androgen receptor gene, or on each side thereof, provided that is on the same chromosome and in linkage disequilibrium with the AR polymorphism of the present invention. Distances between markers in linkage disequilibrium can vary widely (below 50 kb to more than 1 mega base) depending on the genetic structure of the population and is ascertainable by a statistically significant association between the markers.

It shall be recognized by the person skilled in the art to which the present invention pertains, that since some of the polymorphisms herein identified in the AR gene can be within the coding region of the gene and therefore expressed, that the present invention should not be limited to the identification of polymorphisms at the DNA level (whether on genomic DNA, amplified DNA, cDNA or the like). Indeed, the herein-identified polymorphisms could be detected at the mRNA or protein level. Such detections of polymorphism identification on mRNA or protein are known in the art. Non-limiting examples include detection based on oligos designed to hybridize to mRNA or ligands such as antibodies which are specific to the encoded polymorphism (i.e. specific to the protein fragment encoded by the CAG repeat for example).

Since some of the polymorphisms of the present invention are expressed, one of the advantages of the present invention is to enable a determination of the polymorphisms in the AR gene, in easily obtainable cells which express these genes. A non-limiting example thereof is lymphocytes, thereby enabling a genotyping from a simple blood sample.

Nucleotide sequences are presented herein by single strand, in the 5′ to 3′ direction, from left to right, using the one letter nucleotide symbols as commonly used in the art and in accordance with the recommendations of the IUPAC-IUB Biochemical Nomenclature Commission.

Unless defined otherwise, the scientific and technological terms and nomenclature used herein have the same meaning as commonly understood by a person of ordinary skill to which this invention pertains. Generally, the procedures for cell cultures, infection, molecular biology methods and the like are common methods used in the art. Such standard techniques can be found in reference manuals such as for example Sambrook et al. (1989, Molecular Cloning—A Laboratory Manual, Cold Spring Harbor Laboratories) and Ausubel et al. (1994, Current Protocols in Molecular Biology, Wiley, N.Y.).

The present description refers to a number of routinely used recombinant DNA (rDNA) technology terms. Nevertheless, definitions of selected examples of such rDNA terms are provided for clarity and consistency.

As used herein, “nucleic acid molecule”, refers to a polymer of nucleotides. Non-limiting examples thereof include DNA (i.e. genomic DNA, cDNA) and RNA molecules (i.e. mRNA). The nucleic acid molecule can be obtained by cloning techniques or synthesized. DNA can be double-stranded or single-stranded (coding strand or non-coding strand (antisensel).

The term “recombinant DNA” as known in the art refers to a DNA molecule resulting from the joining of DNA segments. This is often referred to as genetic engineering.

The term “DNA segment”, is used herein, to refer to a DNA molecule comprising a linear stretch or sequence of nucleotides. This sequence when read in accordance with the genetic code, can encode a linear stretch or sequence of amino acids which can be referred to as a polypeptide, protein, protein fragment and the like.

The terminology “amplification pair” refers herein to a pair of oligonucleotides (oligos) of the present invention, which are selected to be used together in amplifying a selected nucleic acid sequence by one of a number of types of amplification processes, preferably a polyrrierase chain reaction. Other types of amplification processes include ligase chain reaction, strand displacement amplification, or nucleic acid sequence-based amplification, as explained in greater detail below. As commonly known in the art, the oligos are designed to bind to a complementary sequence under selected conditions.

The nucleic acid (i.e. DNA or RNA) for practicing the present invention may be obtained according to well known methods.

Oligonucleotide probes or primers of the present invention may be of any suitable length, depending on the particular assay format and the particular needs and targeted genomes employed. In general, the oligonucleotide probes or primers are at least 12 nucleotides in length, preferably between 15 and 24 molecules, and they may be adapted to be especially suited to a chosen nucleic acid amplification system. As commonly known in the art, the oligonucleotide probes and primers can be designed by taking into consideration the melting point of hydrizidation thereof with its targeted sequence (see below and in Sambrook et al., 1989, Molecular Cloning—A Laboratory Manual, 2nd Edition, CSH Laboratories; Ausubel et al., 1989, in Current Protocols in Molecular Biology, John Wiley & Sons Inc., N.Y.).

The term “oligonucleotide” or “DNA” molecule or sequence refers to a molecule comprised of the deoxyribonucleotides adenine (A), guanine (G), thymine (T) and/or cytosine (C), in a double-stranded form, and comprises or includes a “regulatory element” according to the present invention, as the term is defined herein. The term “oligonucleotide” or “DNA” can be found in linear DNA molecules or fragments, viruses, plasmids, vectors, chromosomes or synthetically derived DNA. As used herein, particular double-stranded DNA sequences may be described according to the normal convention of giving only the sequence in the 5′ to 3′ direction.

“Nucleic acid hybridization” refers generally to the hybridization of two single-stranded nucleic acid molecules having complementary base sequences, which under appropriate conditions will form a thermodynamically favored double-stranded structure. Examples of hybridization conditions can be found in the two laboratory manuals referred above (Sambrook et al., 1989, supra and Ausubel et al., 1989, supra) and are commonly known in the art. In the case of a hybridization to a nitrocellulose filter, as for example in the well known Southern blotting procedure, a nitrocellulose filter can be incubated overnight at 65° C. with a labeled probe in a solution containing 50% formamide, high salt (5×SSC or 5×SSPE), 5× Denhardt's solution, 1% SDS, and 100 μg/ml denatured carrier DNA (i.e. salmon sperm DNA). The non-specifically binding probe can then be washed off the filter by several washes in 0.2×SSC/0.1% SDS at a temperature which is selected in view of the desired stringency: room temperature (low stringency), 42° C. (moderate stringency) or 65° C. (high stringency). The selected temperature is based on the melting temperature (Tm) of the DNA hybrid. Of course, RNA-DNA hybrids can also be formed and detected. In such cases, the conditions of hybridization and washing can be adapted according to well known methods by the person of ordinary skill. Stringent conditions will be preferably used (Sambrook et al., 1989, supra).

Probes of the invention can be utilized with naturally occurring sugar-phosphate backbones as well as modified backbones including phosphorothioates, dithionates, alkyl phosphonates and α-nucleotides and the like. Modified sugar-phosphate backbones are generally taught by Miller, 1988, Ann. Reports Med. Chem. 23:295 and Moran et al., 1987, Nucleic acid molecule. Acids Res., 14:5019. Probes of the invention can be constructed of either ribonucleic acid (RNA) or deoxyribonucleic acid (DNA), and preferably of DNA.

The types of detection methods in which probes can be used include Southern blots (DNA detection), dot or slot blots (DNA, RNA), and Northern blots (RNA detection). Although less preferred, labeled proteins could also be used to detect a particular nucleic acid sequence to which it binds. More recently, PNAs have been described (Nielsen et al. 1999, Current Opin. Biotechnol. 10:71-75). PNAs could also be used to detect the polymorphisms of the present invention. Other detection methods include kits containing probes on a dipstick setup and the like.

Although the present invention is not specifically dependent on the use of a label for the detection of a particular nucleic acid sequence, such a label might be beneficial, by increasing the sensitivity of the detection. Furthermore, it enables automation. Probes can be labeled according to numerous well known methods (Sambrook et al., 1989, supra). Non-limiting examples of labels include ³H, ¹⁴C, ³²P, and ³⁵S. Non-limiting examples of detectable markers include ligands, fluorophores, chemiluminescent agents, enzymes, and antibodies. Other detectable markers for use with probes, which can enable an increase in sensitivity of the method of the invention, include biotin and radionucleotides. It will become evident to the person of ordinary skill that the choice of a particular label dictates the manner in which it is bound to the probe.

As commonly known, radioactive nucleotides can be incorporated into probes of the invention by several methods. Non-limiting examples thereof include kinasing the 5′ ends of the probes using gamma ³²P ATP and polynucleotide kinase, using the Klenow fragment of Pol I of E. coli in the presence of radioactive dNTP (i.e. uniformly labeled DNA probe using random oligonucleotide primers in low-melt gels), using the SP6/T7 system to transcribe a DNA segment in the presence of one or more radioactive NTP, and the like.

As used herein, “oligonucleotides” or “oligos” define a molecule having two or more nucleotides (ribo or deoxyribonucleotides). The size of the oligo will be dictated by the particular situation and ultimately on the particular use thereof and adapted accordingly by the person of ordinary skill. An oligonucleotide can be synthesized chemically or derived by cloning according to well known methods.

As used herein, a “primer” defines an oligonucleotide which is capable of annealing to a target sequence, thereby creating a double stranded region which can serve as an initiation point for DNA synthesis under suitable conditions.

Amplification of a selected, or target, nucleic acid sequence may be carried out by a number of suitable methods. See generally Kwoh et al., 1990, Am. Biotechnol. Lab. 8:14-25. Numerous amplification techniques have been described and can be readily adapted to suit particular needs of a person of ordinary skill. Non-limiting examples of amplification techniques include polymerase chain reaction (PCR), ligase chain reaction (LCR), strand displacement amplification (SDA), transcription-based amplification, the Qβ replicase system and NASBA (Kwoh et al., 1989, Proc. Natl. Acad. Sci. USA 86, 1173-1177; Lizardi et al., 1988, BioTechnology 6:1197-1202; Malek et al., 1994, Methods Mol. Biol., 28:253-260; and Sambrook et al., 1989, supra). Preferably, amplification will be carried out using PCR.

Polymerase chain reaction (PCR) is carried out in accordance with known techniques. See, e.g., U.S. Pat. Nos. 4,683,195; 4,683,202; 4,800,159; and 4,965,188 (the disclosures of all three U.S. patent are incorporated herein by reference). In general, PCR involves, a treatment of a nucleic acid sample (e.g., in the presence of a heat stable DNA polymerase) under hybridizing conditions, with one oligonucleotide primer for each strand of the specific sequence to be detected. An extension product of each primer which is synthesized is complementary to each of the two nucleic acid strands, with the primers sufficiently complementary to each strand of the specific sequence to hybridize therewith. The extension product synthesized from each primer can also serve as a template for further synthesis of extension products using the same primers. Following a sufficient number of rounds of synthesis of extension products, the sample is analysed to assess whether the sequence or sequences to be detected are present. Detection of the amplified sequence may be carried out by visualization following EtBr staining of the DNA following gel electrophores, or using a detectable label in accordance with known techniques, and the like. For a review on PCR techniques (see PCR Protocols, A Guide to Methods and Amplifications, Michael et al. Eds, Acad. Press, 1990).

Ligase chain reaction (LCR) is carried out,in accordance with known techniques (Weiss, 1991, Science 254:1292). Adaptation of the protocol to meet the desired needs can be carried out by a person of ordinary skill. Strand displacement amplification (SDA) is also carried out in accordance with known techniques or adaptations thereof to meet the particular needs (Walker et al., 1992, Proc. Natl. Acad. Sci. USA 89:392-396; and ibid., 1992, Nucleic Acids Res. 20:1691-1696).

As used herein, the term “gene” is well known in the art and relates to a nucleic acid sequence defining a single protein or polypeptide. A “structural gene” defines a DNA sequence which is transcribed into RNA and translated into a protein having a specific amino acid sequence thereby giving rise the a specific polypeptide or protein.

A “heterologous” (i.e. a heterologous gene) region of a DNA molecule is a subsegment segment of DNA within a larger segment that is not found in association therewith in nature. The term “heterologous” can be similarly used to define two polypeptidic segments not joined together in nature. Non-limiting examples of heterologous genes include reporter genes such as luciferase, chloramphenicol acetyl transferase, β-galactosidase, and the like which can be juxtaposed or joined to heterologous control regions or to heterologous polypeptides.

The term “vector” is commonly known in the art and defines a plasmid DNA, phage DNA, viral DNA and the like, which can serve as a DNA vehicle into which DNA of the present invention can be cloned. Numerous types of vectors exist and are well known in the art.

The term “expression” defines the process by which a gene is transcribed into mRNA (transcription), the mRNA is then being translated (translation) into one polypeptide (or protein) or more.

The terminology “expression vector” defines a vector or vehicle as described above but designed to enable the expression of an inserted sequence following transformation into a host. The cloned gene (inserted sequence) is usually placed under the control of control element sequences such as promoter sequences. The placing of a cloned gene under such control sequences is often refered to as being operably linked to control elements or sequences.

Operably linked sequences may also include two segments that are transcribed onto the same RNA transcript. Thus, two sequences, such as a promoter and a “reporter sequence” are operably linked if transcription commencing in the promoter will produce an RNA transcript of the reporter sequence. In order to be “operably linked” it is not necessary that two sequences be immediately adjacent to one another.

Expression control sequences will vary depending on whether the vector is designed to express the operably linked gene in a prokaryotic or eukaryotic host or both (shuttle vectors) and can additionally contain transcriptional elements such as enhancer elements, termination sequences, tissue-specificity elements, and/or translational initiation and termination sites.

Prokaryotic expressions are useful for the preparation of large quantities of the protein encoded by the DNA sequence of interest. This protein can be purified according to standard protocols that take advantage of the intrinsic properties thereof, such as size and charge (i.e. SDS gel electrophoresis, gel filtration, centrifugation, ion exchange chromatography . . . ). In addition, the protein of interest can be purified via affinity chromatography using polyclonal or monoclonal antibodies. The purified protein can be used for therapeutic applications.

The DNA construct can be a vector comprising a promoter that is operably linked to an oligonucleotide sequence of the present invention, which is in turn, operably linked to a heterologous gene, such as the gene for the luciferase reporter molecule. “Promoter” refers to a DNA regulatory region capable of binding directly or indirectly to RNA polymerase in a cell and initiating transcription of a downstream (3′ direction) coding sequence. For purposes of the present invention, the promoter is bound at its 3′ terminus by the transcription initiation site and extends upstream (5′ direction) to include the minimum number of bases or elements necessary to initiate transcription at levels detectable above background. Within the promoter will be found a transcription initiation site (conveniently defined by mapping with S1 nuclease), as well as protein binding domains (consensus sequences) responsible for the binding of RNA polymerase. Eukaryotic promoters will often, but not always, contain “TATA” boses and “CCAT” boxes. Prokaryotic promoters contain Shine-Dalgamo sequences in addition to the −10 and −35 consensus sequences.

In accordance with one embodiment of the present invention, an expression vector can be constructed to assess the functionality of specific alleles of the AR gene and of the interaction of such alleles. Non-limiting examples of such expression vectors include a vector comprising the androgen responsive element (the cis sequences [i.e. DNA sequence to which a factor binds] enabling androgen-dependent modulating effects of promoter activity are known in the art) operably linked to a chosen promoter and modulating the activity thereof, the promoter driving the expression of a reporter gene. When such a vector is tranfected in a cell expressing AR, the modulating effect of the promoter activity can be assessed by determining the level of expression of the reporter gene. In one embodiment, the vector is transfected into a cell of a patient having the genotype of AR shown herein to be associated with a low risk of breast cancer, or in a cell from a patient having the genotype of AR shown herein to be associated with a moderate or high risk of breast cancer. These cells can serve to screen for compounds that modulate the promoter activity, in order to identify compounds that could be used to treat especially, patients predicted to be at moderate or high risk of breast cancer. Of course, it will be understood that the AR gene expressed by these cells can be modified at will (i.e. by in vitro mutagenesis or the like). Similarly, numerous combinations of genotypes can be tested in such assays to dissect the functional relationship between the AR genotype and its function in androgen-dependent function and/or its function in breast cancer. It will also be clear to the skilled artisan, that such indicator cells expressing AR, could also be engineered by choosing a cell line and transfecting thereinto, chosen genotypes of AR and one expression vector as described above. Non-human transgenic animals expressing chosen alleles of AR could also be prepared and used to screen compounds that affect androgen receptor function and possibly overcome a predisposition to breast cancer.

As used herein, the designation “functional derivative” denotes, in the context of a functional derivative of a sequence whether an nucleic acid or amino acid sequence, a molecule that retains a biological activity (either function or structural) that is substantially similar to that of the original sequence. This functional derivative or equivalent may be a natural derivative or may be prepared synthetically. Such derivatives include amino acid sequences having substitutions, deletions, or additions of one or more amino acids, provided that the biological activity of the protein is conserved. The same applies to derivatives of nucleic acid sequences which can have substitutions, deletions, or additions of one or more nucleotides, provided that the biological activity of the sequence is generally maintained. When relating to a protein sequence, the substituting amino acid as chemico-physical properties which are similar to that of the substituted amino acid. The similar chemico-physical properties include, similarities in charge, bulkiness, hydrophobicity, hydrophylicity and the like. The term “functional derivatives” is intended to include “fragments”, “segments”, “variants”, “analogs” or “chemical derivatives” of the subject matter of the present invention.

Thus, the term “variant” refers herein to a protein or nucleic acid molecule which is substantially similar in structure and biological activity to the protein or nucleic acid of the present invention.

The functional derivatives of the present invention can be synthesized chemically or produced through recombinant DNA technology all these methods are well known in the art.

As used herein, “chemical derivatives” is meant to cover additional chemical moieties not normally part of the subject matter of the invention. Such moieties could affect the physico-chemical characteristic of the derivative (i.e. solubility, absorption, half life and the like, decrease of toxicity). Such moieties are exemplified in Remington's Pharmaceutical Sciences (1980). Methods of coupling these chemical-physical moieties to a polypeptide are well known in the art.

The term “allele” defines an alternative form of a gene which occupies a given locus on a chromosome.

As commonly known, a “mutation” is a detectable change in the genetic material which can be transmitted to a daughter cell. As well known, a mutation can be, for example, a detectable change in one or more deoxyribonucleotide. For example, nucleotides can be added, deleted, substituted for, inverted, or transposed to a new position. Spontaneous mutations and experimentally induced mutations exist. The result of a mutations of nucleic acid molecule is a mutant nucleic acid molecule. A mutant polypeptide can be encoded from this mutant nucleic acid molecule.

As used herein, the term “purified” refers to a molecule having been separated from a cellular component. Thus, for example, a “purified protein” has been purified to a level not found in nature. A “substantially pure” molecule is a molecule that is lacking in all other cellular components.

As used herein, the terms “molecule”, “compound”, or “agent” are used interchangeably and broadly to refer to natural, synthetic or semi-synthetic molecules or compounds. The term “molecule” therefore denotes for example chemicals, macromolecules, cell or tissue extracts (from plants or animals) and the like. Non limiting examples of molecules include nucleic acid molecules, peptides, ligands, including antibodies, carbohydrates and pharmaceutical agents. The agents can be selected and screened by a variety of means including random screening, rational selection and by rational design using for example protein or ligand modelling methods such as computer modelling. The terms “rationally selected” or “rationally designed” are meant to define compounds which have been chosen based on the configuration of the interaction domains of the present invention. As will be understood by the person of ordinary skill, macromolecules having non-naturally occurring modifications are also within the scope of the term “molecule”. For example, peptidomimetics, well known in the pharmaceutical industry and generally referred to as peptide analogs can be generated by modelling as mentioned above. Similarly, in a preferred embodiment, the polypeptides of the present invention are modified to enhance their stability. It should be understood that in most cases this modification should not alter the biological activity of the protein. The molecules identified in accordance with the teachings of the present invention have a therapeutic value in diseases or conditions in which a apparently lower activity and/or level of the AR is linked to a genotype of AR identified in accordance with the present invention. Alternatively, the molecules identified in accordance with the teachings of the present invention find utility in the development of compounds which can modulate the activity and/or level of the androgen receptor in an animal and/or overcome a predisposition to breast cancer.

As used herein, agonists and antagonists also include potentiators of known compounds with such agonist or antagonist properties. In one embodiment, modulators of the level or the activity of the AR can be identified and selected by contacting the indicator cell with a compound or mixture or library of molecules for a fixed period of time. In certain embodiments, the “breast cancer-low risk-associated alleles” of the AR gene can be used as positive controls.

An indicator cell in accordance with the present invention can be used to identify antagonists. For example, the test molecule or molecules are incubated with the host cell in conjunction with one or more agonists held at a fixed concentration. An indication and relative strength of the antagonistic properties of the molecule(s) can be provided by comparing the level of gene expression in the indicator cell in the presence of the agonist, in the absence of test molecules vs in the presence thereof. Of course, the antagonistic effect of a molecule can also be determined in the absence of agonist, simply by comparing the level of expression of the reporter gene product in the presence and absence of the test molecule(s).

It shall be understood that the “in vivo” experimental model can also be used to carry out an “in vitro” assay. For example, cellular extracts from the indicator cells can be prepared and used in an “in vitro” test. A non-limiting example thereof include binding assays.

As used herein the recitation “indicator cells” refers to cells that express a given genotype of AR according to the present invention. As alluded to above, such indicator cells can be used in the screening assays of the present invention. In certain embodiments, the indicator cells have been engineered so as to express a chosen derivative, fragment, homolog, or mutant of a genotype of the present invention. The cells can be yeast cells or higher eukaryotic cells such as mammalian cells. In one particular embodiment, the indicator cell would be a yeast cell harboring vectors enabling the use of the two hybrid system technology, as well known in the art (Ausubel et al., 1994, supra) and can be used to test a compound or a library thereof. In another embodiment, the cis-trans assay as described in U.S. Pat. No. 4,981,784, can be adapted and used in accordance with the present invention. Such an indicator cell could be used to rapidly screen at high-throughput a vast array of test molecules. In a particular embodiment, the reporter gene is luciferase or β-Gal.

In some embodiments, it might be beneficial to express a fusion protein. The design of constructs therefor and the expression and production of fusion proteins and are well known in the art (Sambrook et al., 1989, supra; and Ausubel et al., 1994, supra).

Non limiting examples of such fusion proteins include a hemaglutinin fusions and Gluthione-S-transferase (GST) fusions and Maltose binding protein (MBP) fusions. In certain embodiments, it might be beneficial to introduce a protease cleavage site between the two polypeptide sequences which have been fused. Such protease cleavage sites between two heterologously fused polypeptides are well known in the art.

In certain embodiments, it might also be beneficial to fuse the protein of the present invention to signal peptide sequences enabling a secretion of the fusion protein from the host cell. Signal peptides from diverse organisms are well known in the art. Bacterial OmpA and yeast Suc2 are two non limiting examples of proteins containing signal sequences. In certain embodiments, it might also be beneficial to introduce a linker (commonly known) between the interaction domain and the heterologous polypeptide portion. Such fusion protein find utility in the assays of the present invention as well as for purification purposes, detection purposes and the like.

For certainty, the sequences and polypeptides useful to practice the invention include without being limited thereto mutants, homologs, subtypes, alleles and the like. It shall be understood that generally, the sequences of the present invention should encode a functional (albeit defective) AR. It will be clear to the person of ordinary skill that whether the AR sequence of the present invention, variant, derivative, or fragment thereof retains its function, can be determined by using the teachings and assays of the present invention and the general teachings of the art.

It should be understood that the AR protein of the present invention can be modified, for example by in vitro mutagenesis, to dissect the structure-function relationship thereof and permit a better design and identification of modulating compounds. However, some derivative or analogs having lost their biological function may still find utility, for example for raising antibodies. These antibodies could be used for detection or purification purposes. In addition, these antibodies could also act as competitive or non-competitive inhibitor and be found to be modulators of the activity of the AR protein of the present invention.

A host cell or indicator cell has been “transfected” by exogenous or heterologous DNA (e.g. a DNA construct) when such DNA has been introduced inside the cell. The transfecting DNA may or may not be integrated (covalently linked) into chromosomal DNA making up the genome of the cell. In prokaryotes, yeast, and mammalian cells for example, the transfecting DNA may be maintained on a episomal element such as a plasmid. With respect to eukaryotic cells, a stably transfected cell is one in which the transfecting DNA has become integrated into a chromosome so that it is inherited by daughter cells through chromosome replication. This stability is demonstrated by the ability of the eukaryotic cell to establish cell lines or clones comprised of a population of daughter cells containing the transfecting DNA. Transfection methods are well known in the art (Sambrook et al., 1989, supra; Ausubel et al., 1994 supra). The use of a mammalian cell as indicator can provide the advantage of furnishing an intermediate factor, which permits for example the interaction of two polypeptides which are tested, that might not be present in lower eukaryotes or prokaryotes. It will be understood that extracts from mammalian cells for example could be used in certain embodiments, to compensate for the lack of certain factors.

In general, techniques for preparing antibodies (including monoclonal antibodies and hybridomas) and for detecting antigens using antibodies are well known in the art (Campbell, 1984, In “Monoclonal Antibody Technology: Laboratory Techniques in Biochemistry and Molecular Biology”, Elsevier Science Publisher, Amsterdam, The Netherlands) and in Harlow et al., 1988 (in: Antibody-A Laboratory Manual, CSH Laboratories). The present invention also provides polyclonal, monoclonal antibodies, or humanized versions thereof, chimeric antibodies and the like which inhibit or neutralize their respective interaction domains and/or are specific thereto.

From the specification and appended claims, the term therapeutic agent should be taken in a broad sense so as to also include a combination of at least two such therapeutic agents. Further, the DNA segments or proteins according to the present invention could be introduced into individuals in a number of ways. For example, cells can be isolated from the afflicted individual, transformed with a DNA construct according to the invention and reintroduced to the afflicted individual in a number of ways. Alternatively, the DNA construct can be administered directly to the afflicted individual. The DNA construct can also be delivered through a vehicle such as a liposome, which can be designed to be targeted to a specific cell type, and engineered to be administered through different routes. For example, an androgen receptor gene having the genotype associated with low risk of breast cancer could be introduced in cells or in an individual displaying the AR polymorphism associated with high risk of breast cancer.

For administration to humans, the prescribing medical professional will ultimately determine the appropriate form and dosage for a given patient, and this can be expected to vary according to the chosen therapeutic regimen (i.e. DNA construct, protein, cells), the response and condition of the patient as well as the severity of the disease.

Composition within the scope of the present invention should contain the active agent (i.e. molecule, hormone) in an amount effective to achieve the desired therapeutic effect while avoiding adverse side effects. Typically, the nucleic acids in accordance with the present invention can be administered to mammals (i.e. humans) in doses ranging from 0.005 to 1 mg per kg of body weight per day of the mammal which is treated. Pharmaceutically acceptable preparations and salts of the active agent are within the scope of the present invention and are well known in the art (Remington's Pharmaceutical Science, 16th Ed., Mack Ed.). For the administration of polypeptides, antagonists, agonists and the like, the amount administered should be chosen so as to avoid adverse side effects. The dosage will be adapted by the clinician in accordance with conventional factors such as the extent of the disease and different parameters from the patient. Typically, 0.001 to 50 mg/kg/day will be administered to the mammal.

The present invention relates to a kit for assessing a predisposition to breast cancer comprising a determination of the genotype at the AR locus (or a locus in linkage desiquilibrium therewith) using a nucleic acid fragment, a protein or a ligand, or a restriction enzyme in accordance with the present invention. For example, a compartmentalized kit in accordance with the present invention includes any kit in which reagents are contained in separate containers. Such containers include small glass containers, plastic containers or strips of plastic or paper. Such containers allow the efficient transfer of reagents from one compartment to another compartment such that the samples and reagents are not cross-contaminated and the agents or solutions of each container can be added in a quantitative fashion from one compartment to another. Such containers will include in one particular embodiment a container which will accept the test sample (DNA protein or cells), a container which contains the primers used in the assay, containers which contain enzymes, containers which contain wash reagents, and containers which contain the reagents used to detect the extension products.

It will be readily recognized by the person of ordinary skill, that the nucleic acid sequences, probes, primers, antibodies and the like of the present invention enabling a detection of the CAG repeat polymorphism of the AR gene of the present invention can be incorporated into anyone of numerous established kit formats which are well known in the art.

Other objects, advantages and features of the present invention will become more apparent upon reading of the following non-restrictive description of preferred embodiments which is exemplary and should not be interpreted as limiting the scope of the present invention.

### DESCRIPTION OF THE PREFERRED EMBODIMENT

In accordance with one embodiment of the invention, there is provided a specific model for use in prediction of breast cancer susceptibility and prognosis. The model comprises an androgen receptor gene polymorphism that allows to identify a subset of patients (i.e. women) that are at significantly increased risk of breast cancer as compared to those bearing other variants of this gene.

In accordance with a preferred embodiment of the present invention, a single gene, the androgen receptor gene, has been identified. The polymorphism of this gene is associated with a significant proportion of breast cancer cases in the general population (up to 60% of all cases). Polymorphism of this gene is for example the CAG repeat located in the first exon.

It was thus discovered in accordance with a preferred embodiment of the present invention that testing for this polymorphism in the androgen receptor (AR) gene allows to distinguish between women at lower risk of breast cancer and those at higher risk of the disease.

The present invention will be more readily understood by referring to the following example which is given to illustrate the invention rather than to limit its scope.

### EXAMPLE 1

### Polymorphism of the CAG Repeat of the Androgen Receptor as a Marker for Breast Cancer Susceptibility

In a case control study comparing 262 consecutive cases of breast cancer in women and 465 control women matched for age, polymorphism at the AR gene, namely, the CAG repeat coding for a polyglutamine tract in the 5′ part of the AR gene located on chromosome X, was studied. Because of the large number of alleles identified (15 different alleles), these alleles were grouped arbitrarily in categories by size to simplify the analysis and increase the number of individuals in each category. Table 1 presents the frequency of cases and controls in categories of genotypes with the corresponding odds ratio for breast cancer and the computed 95% confidence intervals. The AR gene alleles were called arbitrarily A to E according to their size in CAG repeats, the shortest alleles being A and the longest being called E. The shortest AR gene alleles (corresponding to the polyglutamine stretch) or combinations of short alleles (AA,AB,BB) are the genotypes that show the smallest breast cancer risk. This shows that women with a certain combination of AR gene polymorphisms on their two X chromosomes have a significantly increased risk of developing breast cancer as compared to the category with the smallest risk. In fact, in this cohort 32% of all cases of breast cancer were attributable to variation in the AR gene. This is three to six times the number of breast cancer cases attributable to the BRCA1 and BRCA2 genes. Indeed in the cohort studied the 25% of women with the AR genotypes associated with the smallest risk of breast cancer comprised only 19% of all breast cancer cases while the 75% of women having the. AR genotypes associated with the highest risks of breast cancer had 81% of all breast cancer cases. In other words, as compared with the general population, for which the risk of breast cancer is of 1:9 women, women with certain AR genotypes had a risk of 1:12 (much lower; i.e. protecting effect) while the other group had a risk of 1:8 (larger). Thus, this novel genetic marker of breast cancer allows to identify a subgroup of women with a risk of breast cancer close to two times larger than the other subgroup.

TABLE 1
Distribution of cases and controls among
females with various AR genotypes

| AR genotype | Cases | Controls | Totals |
|---|---|---|---|
| A* + BB | 49 | 134 | 183 |
| BC to EE | 213 | 331 | 544 |
| Totals | 262 | 465 | 727 |

Odds Ratio (OR) for breast cancer in BC to EE genotypes vs A* and BB = 1.76 (95% confidence interval CI 1.22 to 2.55)
Chi-square = 9.5
p = 0.002
Breast cancer risk attributable to AR gene variation = 32% (57 cases/213 in the others category)

As will be clear to the skilled artisan, the different alleles AR alleles can be grouped differently according to size, and the invention should therefore not be limited to particular groupings. As will be seen in Table 2, groupings of the alleles in three categories instead of 5, still enable a demonstration of the significant association of the AR CAG-repeat polymorphism with breast cancer.

In Table 2, the 15 different alleles were grouped in three different categories (X, Y, and Z) instead of five, in which the shortest alleles are in the X category, and the longest alleles are in the Z category. The six possible genotypes were thus designated as “XX”, “XY”, “XZ”, “YY”, “YZ”, and “Zz” genotypes. It is apparent from Table 2 that (CAG)n genotypes were associated with the disease as the genotypes with mid to large numbers of (CAG) repeats were at significantly higher risk of developing the disease as compared to genotypes with shorter (CAG)n tracts (Table 2). Table 2 shows that women with either the YY, YZ or ZZ genotypes had a 2.2-fold increased risk of breast cancer compared to women with the XX or XY genotype, i.e. that women with these later genotypes had only a 1:20 lifetime risk for the disease as compared to a 1:9 risk for those with the larger genotypes.

TABLE 2
Association of androgen receptor
polymorphism with breast cancer

| | (CAG)n genotype | | |
|---|---|---|---|
| | XX or XY | | YY, YZ or ZZ |
| | genotype | XZ genotype | genotype |
| Cases | 10 (4%)* | 28 (11%)* | 212 (85%)* |
| Controls | 37 (8%)* | 61 (13%)* | 355 (78%)* |
| Odds Ratio (OR) | 1.0 | 1.7 | 2.2 |
| 95% CI for OR | | 0.7-3.9 | 1.1-4.5 |
| (min-max) | | | |
| Lifetime risk of | 1:20 | 1:12 | 1:9 |
| breast cancer | | | |

*value in parenthesis represents percentage of total cases or controls
CI Confidence interval expressed with the highest and lowest values.

No significant interaction was observed between AR genotypes and the body mass index (BMI), smoking habits, menopausal status or family history of breast cancer. However, a striking combined influence of the AR genotype with a positive history of breast benign disease (BBD) on the risk of breast cancer was observed (Table 3). Women with a positive history of BBD and AR genotypes combining the large AR alleles (Y or Z) had a relative risk of 3.5 as compared to women with no such history and AR genotypes comprised of smaller alleles. When compared to carriers of XX, XY AR genotypes only (who have the lowest risk of breast cancer) with no history of benign disease, women with the AR-ZZ genotype had an odds ratio of 7.1 for breast cancer (95% Cl 2.3 to 22). Interestingly the AR genotype was not associated with a significant risk of breast cancer in women with no history of breast benign disease. The present invention thus also provides as an additional “marker” to strengthen the prognosis/diagnosis/treatment methods and reagents according to the present invention, a positive history of BBD.

TABLE 3
Association of breast cancer risk with AR
polymorphism and breast benign disease

| | | AR genotype | |
|---|---|---|---|
| | | XX, XY, XZ | YY, YZ, ZZ |
| Negative history | Cases | 25 (10%)* | 131 (52%)* |
| of benign breast | Controls | 73 (16%)* | 288 (64%)* |
| disease | Odds Ratio | 1.0 | 1.33 |
| | 95% CI for OR | — | 0.8-2.2 |
| | (min-max) | | |
| | Lifetime risk of | 1:16 | 1:12 |
| | breast cancer | | |
| Positive history | Cases | 13 (5%)* | 81 (32%)* |
| of benign breast | Controls | 25 (6%)* | 67 (15%)* |
| | Odds Ratio | 1.5 | 3.5 |
| | 95% CI for OR | 0.7-3.4 | 2.0-6.2 |
| | (min-max) | | |
| | Lifetime risk of | 1:11 | 1:4 |
| | breast cancer | | |

*value in parenthesis represents percentage of total cases or controls
CI Confidence interval expressed with the highest and lowest values.

Up to now, no marker displaying such a large odds ratios had been reported for breast cancer. Furthermore, this genetic marker and polymorphisms in the AR gene play a very significant role in breast cancer susceptibility in women, as evidenced by the very significant association demonstrated herein. The present invention also points to alternative therapies for breast cancer aiming at restoring the efficacy of the AR in women with a reduced function of their AR genes due to the variant genotypes that they carry. The described assays of the present invention could enables the identification of such therapies.

Although the present invention has been described hereinabove by way of preferred embodiments thereof, it can be modified, without departing from the spirit and nature of the subject invention as defined in the appended claims.

1. A method of determining an individual's predisposition to breast cancer, development of breast cancer and/or responsiveness to therapy for breast cancer, said method comprising the step of determining a polymorphism at the CAG repeat of the androgen receptor (AR) gene or a DNA variant equivalent, or mutation which shows a linkage disequilibrium therewith, whereby said polymorphism at the AR gene, or marker in linkage disequilibrium therewith enables a prediction of an individual's predisposition to breast cancer, development of breast cancer and/or responsiveness to therapy for breast cancer.

2. The method of claim 1, wherein the androgen receptor genotype is determined by determining the number of CAG repeats within the androgen receptor gene

3. The method of claim 2, which further comprises a step of amplifying a segment of the androgen receptor using polymerase chain reaction.

4. The method of claim 3, wherein a pair of primers derived from a nucleic acid sequence of the androgen receptor gene or flanking said gene is used in the polymerase chain reaction.

5. The method of claim 4, wherein the segment of the androgen receptor gene is amplified using a pair of primers as follows:

| | |
|---|---|
| 5′-TCCAGAATCT GTTCCAGAGC GTGC-3′; | SEQ ID NO: 1 |
| and | |
| 5′-GCTGTGAAGG TTGCTGTTCC TCAT-3′. | SEQ ID NO: 2 |

6. The method according to claim 1, wherein said polymorphism at the AR gene, or marker in linkage disequilibrium therewith, is determined from DNA obtained from said individual.

7. The method of claim 6, wherein said DNA is genomic DNA.

8. The method according to claim 7, wherein said DNA is obtained from non-cancerous cells.

9. The method of claim 8, wherein said cell is obtained from a tissue or blood sample.

10. An assay for screening and selecting an agent which modulates breast cancer predisposition comprising:
a) a recombinant androgen receptor (AR) gene or functional fragment thereof, which comprises a CAG repeat polymorphism in exon 1 thereof, or a marker in linkage disequilibrium therewith; and
b) assaying a function of said androgen receptor;
wherein an allele which modulates said function of said androgen receptor can be selected, and wherein a modulation of a function of said androgen receptor is associated with a modulation of said breast cancer predisposition, whereby short CAG repeats of said AR positively modulate androgen receptor function, while long CAG repeats of said AR negatively modulate Androgen receptor function, thereby leading to breast cancer protection or breast cancer predisposition.

11. An assay for screening and selecting an agent which modulates breast cancer predisposition comprising:
a) an expression vector comprising a promoter operably linked to a reporter gene, said promoter comprising an androgen response element, said response element affecting the activity of said promoter upon binding thereto of androgen or analog thereof;
b) a cell expressing a chosen allele of an androgen receptor and harboring said vector of a);
c) submitting said cell to at least one agent; and
d) assaying a level of said reporter gene;
whereby an agent which modulates breast cancer predisposition can be selected when the level of said reporter gene is significantly modulated by the presence of said agent through its action through the androgen receptor.

12. A method for screening and selecting an agent which can modulate breast cancer predisposition comprising:
a) selecting a specific allele of the androgen receptor (AR) gene, variant, equivalent, or mutation thereof which shows linkage disequilibrium therewith;
b) assaying a function of said AR allele of a); and
c) selecting an agent which can modulate breast cancer predisposition,
wherein an agent which modulates AR function is selected as an agent capable of modulating breast cancer predisposition when said function is significantly different in the presence of said agent, as compared to in the absence thereof.

13. The method of claim 1, wherein the shortest alleles or a combination thereof are associated with a protection to breast cancer, and the intermediate to large alleles or a combination of the intermediate and largest alleles are associated with a predisposition to breast cancer.

14. The method of claim 12, wherein said assay is a cis-trans assay.

15. The method of claim 2, wherein the shortest alleles or a combination thereof are associated with a protection to breast cancer, and the intermediate to large alleles or a combination of the intermediate and largest alleles are associated with a predisposition to breast cancer.

16. The method of claim 6, wherein the shortest alleles or a combination thereof are associated with a protection to breast cancer, and the intermediate to large alleles or a combination of the intermediate and largest alleles are associated with a predisposition to breast cancer.

17. The method of claim 8, wherein the shortest alleles or a combination thereof are associated with a protection to breast cancer, and the intermediate to large alleles or a combination of the intermediate and largest alleles are associated with a predisposition to breast cancer.

18. The method of claim 12, wherein the shortest alleles or a combination thereof are associated with a protection to breast cancer, and the intermediate to large alleles or a combination of the intermediate and largest alleles are associated with a predisposition to breast cancer.
